SEQUENCE LISTING (3 sequences)

SEQ ID NO: 1 - DNA, 27 bp, Artificial Sequence
  Synthetic
gacatcatggagctatagagcgaacag

SEQ ID NO: 2 - DNA, 15 bp, Artificial Sequence
  Synthetic
ctatagagcgaacag

SEQ ID NO: 3 - DNA, 23 bp, Artificial Sequence
  Synthetic
agctgcatcggatcctacgctcg

(19) United States
(12) Patent Application Publication
(10) Pub. No.: US 2024/0240234 A1
(43) Pub. Date: Jul. 18, 2024

(54) METHODS FOR MEASURING PROTEIN-DNA INTERACTIONS WITH LONG-READ DNA SEQUENCING

(71) Applicants: THE REGENTS OF THE UNIVERSITY OF CALIFORNIA, Oakland, CA (US); THE BOARD OF TRUSTEES OF THE LELAND STANFORD JUNIOR UNIVERSITY, Stanford, CA (US); CHAN ZUCKERBERG BIOHUB, INC., San Francisco, CA (US)

(72) Inventors: Aaron Streets, Oakland, CA (US); Nicolas Altemose, Oakland, CA (US); Annie Maslan, Oakland, CA (US); Aaron Straight, Stanford, CA (US); Owen Smith, Stanford, CA (US); Kousik Sundararajan, Stanford (JP)

(21) Appl. No.: 18/564,908

(22) PCT Filed: Jun. 2, 2022
(86) PCT No.: PCT/US22/31869

§ 371 Date: Nov. 28, 2023

Related U.S. Application Data
(60) Provisional application No. 63/196,493, filed on Jun. 3, 2021.

(51) Int. Cl. C12Q 1/6806; C12Q 1/48; C12Q 1/6869

(52) U.S. Cl. CPC C12Q 1/6806; C12Q 1/48; C12Q 1/6869

(57) ABSTRACT

The present disclosure provides materials and methods for mapping specific protein-DNA interactions genome-wide, including highly repetitive areas of the genome, by performing targeted modifications of base-pairs at or near the genomic site where a protein of interest is interacting, followed by direct detection of those modified base-pairs using long-read DNA sequencing.

### STATEMENT REGARDING FEDERALLY SPONSORED RESEARCH

The invention was made with Government support under Grant Nos. GM074728 and GM124916 awarded by NIH National Institute of General Medical Sciences. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “56626_Seqlisting.txt”, which was created on Jun. 2, 2022 and is 885 bytes in size. The subject matter of the Sequence Listing is incorporated herein in its entirety by reference.

### FIELD

The present disclosure relates generally to methods for identifying and measuring protein-DNA interactions.

### BACKGROUND

The interactions between proteins and DNA in the nucleus define the epigenetic state of cells and determine how the genome is regulated. It is therefore important to map where specific protein-DNA interactions are occurring in the genome to understand regulatory processes that guide development, disease, and the everyday functioning of cells in our body. Genome-wide measurement of protein-DNA interactions typically relies on methods which enrich genomic DNA for regions that are actively interacting with or bound to a protein of interest, determining the sequence of those DNA molecules with high-throughput DNA sequencing, and then mapping those sequences back to the reference genome of the organism in question. Standard high-throughput DNA sequencing platforms provide highly accurate sequencing of DNA molecules of limited length, typically around 200 base pairs in length. While these platforms are robust and accurate, their limited read length prevents querying the most repetitive parts of the genome.

For the last 20 years, human genome assembly efforts have excluded 5-10% of the genome, composed mostly of repetitive sequences localized to centromeres, telomeres, and rDNA loci. These regions cannot be confidently assembled because assembly algorithms rely on stitching together uniquely overlapping sequencing reads; if read lengths are short, then unique overlaps cannot be found in repetitive regions, and those regions remain unassembled. Though these regions play essential roles in chromosome segregation, nuclear organization, and transcriptional regulation, studies of repetitive heterochromatic regions have fallen behind the rapid advances of the genomics era, and many fundamental questions remain about their regulation, function, and evolution. For example, among the largest and least understood regions of the human genome are massive arrays of repetitive DNA that occur adjacent to a subset of centromeres. These regions are now being assembled for the first time thanks to the maturation of long-read sequencing technologies (Miga et al., (2020), Nature, 585(7823), 79-84). Because current methods for genome-wide measurement of protein-DNA interactions rely on short-read sequencing, there is no way to confidently map protein-DNA interactions in highly repetitive regions of the genome.

### SUMMARY OF THE INVENTION

One embodiment of the present disclosure provides a method of determining the genomic location of at least one biomolecule-genomic DNA interaction, said method comprising the steps of: (a) incubating a biomolecule of interest under conditions that allow the biomolecule of interest to contact a genomic DNA sequence; (b) isolating and permeabilizing nuclei from the cells in (a) under conditions that allow isolation of genomic DNA bound by the biomolecule of interest; (c) contacting the biomolecule bound to genomic DNA with a first binding moiety capable of specifically binding to the biomolecule of interest; (d) contacting the first binding moiety with a second binding moiety capable of specifically binding to the first binding moiety, wherein said second binding moiety is conjugated to an enzyme capable of modifying genomic DNA; (c) incubating the first binding moiety and second binding moiety of (d) under conditions that allow modification of genomic DNA; (f) isolating and preparing the genomic DNA for sequencing, wherein said preparing does not require amplification of the DNA; and (g) sequencing the genomic DNA under conditions that allow determining the location of the biomolecule-DNA interaction.

In another embodiment, the enzyme capable of modifying genomic DNA is a DNA methyltransferase. In some embodiments, the DNA methyltransferase is selected from the group consisting of DNA adenine methyltransferase (Dam) or a biologically active fragment thereof, (ii) EcoGII methyltransferase or a biologically active fragment thereof, Hia5 or a biologically active fragment thereof, M.CviPI or a biologically active fragment thereof, and M.SssI or a biologically active fragment thereof. In one embodiment, the DNA methyltransferase is Hia5 or a biologically active fragment thereof.

In still other embodiments, an aforementioned method is provided wherein the sequencing conditions of step (g) allow sequencing of more than approximately 1,000 base pairs (bp) in a single sequencing read. In other embodiments, the interaction is in a cell and the incubating of step (a) comprises incubating a collection of cells. In yet other embodiments, the first binding moiety is an antibody or any biological molecule (protein or nucleic acid) capable of binding specifically to the protein of interest and that, once bound, is capable of being bound as described herein. In some embodiments of the present disclosure, the second binding moiety is an antibody, protein-A, protein-G, or protein-A/G.

In yet other embodiments of the present disclosure, the cell is selected from the group consisting of a bacterial cell, a eukaryotic cell, prokaryotic cell, an archacal cell and a virus. In one embodiment, the cell is a mammalian cell. In another embodiment, the cell is a human cell.

The present disclosure also provides an aforementioned method, wherein the biomolecule of interest is selected from the group consisting of a protein, a RNA, and a RNA-DNA hybrid. In some embodiments, the biomolecule is a RNA selected from the group consisting of ncRNA, tRNA, rRNA, snRNA, snoRNA, miRNA, mRNA, and TERC. In still other embodiments, the biomolecule is a protein selected from the group consisting of a nuclear lamina protein, a nucleolar protein, a transcription factor, a histone or histone variant, centromere protein A, an intracellular scFV, a chromatin-modifying enzyme, an RNA polymerase, a DNA polymerase, a DNA helicase, a DNA repair protein, a Cas9 protein, a dCas9 protein, a zinc finger protein, a TALE protein, a CTCF protein, a cohesion protein, a synaptonemal complex protein, a telomere-binding protein, a centromere-binding protein, an outer kinetochore protein, a splicing protein and a chromatin remodeling protein. In another embodiment, the collection of cells are induced to express the protein of interest. In yet another embodiment, the protein of interest is a recombinant protein and is expressed from an expression vector.

In some embodiments, of the present disclosure, an aforementioned method is provided wherein 2, 3, 4, 5, 6, 7, 8, 9 or 10 or more biomolecule-genomic DNA interactions are determined. In some embodiments the modifying genomic DNA of step (e) comprises modifying one or more nucleotides at one or more locations selected from the group consisting of (a) within 1-50 nucleotides of the genomic DNA binding site of the biomolecule, (b) topologically near the genomic DNA binding site of the biomolecule, and (c) both (a) and (b).

In still other embodiments, the isolating and permeabilizing nuclei of step (b) comprises contacting nuclei with digitonin. In some embodiments, the contacting the second binding moiety of step (d) is Protein A. In yet other embodiments, the incubating of step (c) comprises incubating in the presence of bovine serum albumin (BSA) and low salt conditions. In other embodiments, the isolating and preparing the genomic DNA for sequencing of step (f) comprises high molecular weight DNA extraction. In still other embodiments, the sequencing of step (g) comprises long read sequencing.

### BRIEF DESCRIPTION OF THE DRAWINGS

FIG. 1 shows a schematic of the DiMeLo-Seq experimental pipeline. FIG. 1A) Nuclei are permeabilized and a primary antibody, targeting the protein of interest, is allowed to bind its target in situ. FIG. 1B) The pA-Hia5 fusion complex, or similar, is directed to the protein of interest and allowed to methylate adenines in the vicinity of the binding site. FIG. 1C) Genomic DNA, still containing the site-specific methylation is extracted, purified, and sequenced on a long-read sequencing platform such as the ONT minION or the PacBio Sequel, which can directly detect base-modifications on long reads. FIG. 1D) Protein interactions footprints are mapped to complete genome assemblies.

FIG. 2 shows in vitro digestion assay. (FIG. 2A) Two complementary 90-bp oligos are annealed and then treated with an adenine methyltransferase. A single GATC motif at bases 44-47 is the key sequence for determining methylation. If fully methylated or hemimethylated, that GATC site is protected from DpnII digestion; if unmethylated, DpnII digestion occurs. In this way, the fraction of fully intact 90-bp fragments following DpnII digestion indicates the degree of methylation. A Tapestation instrument is used to separate digested DNA molecules by size and quantify the relative intensities of each band. (FIG. 2B) Table of conditions with fragment bands displayed on the left, and computed methylation efficiency reported on the right.

FIG. 3 shows detection of methylation following the in situ protocol targeting LMNB1. Rows 1-5 describe the conditions tested. Row 6 is a negative control, and row 7 is the reference methylation in cells expressing EcoGII-LMNB1. The last column shows the mA signal normalized to the reference. The MethylFlash™ m6A DNA Methylation ELISA Kit (Colorimetric) contains all reagents necessary for the quantification of m6A in DNA. In this assay, DNA is bound to strip wells using DNA high binding solution. m6A is detected using capture and detection antibodies. The detected signal is enhanced and then quantified colorimetrically by reading the absorbance in a microplate spectrophotometer. The amount of m6A is proportional to the OD intensity measured. Image of wells is shown to the left of the last column.

FIG. 4 shows immunofluorescence images of DiMeLo-seq-treated nuclei, verifying proper localization of the methyltransferase construct to the nuclear lamina under various conditions.

FIG. 5 shows a comparison of targeted methylation among short-read DamID, long-read in vivo DiMeLo-seq, and long-read in situ DiMeLo-seq. Sample descriptions are in accompanying table herein. Samples 1-6 are methylation targeted to the nuclear lamina, while samples 7-14 are untargeted, IgG, and no SAM controls. cLAD/ciLAD ratios plotted for long-read samples are at a modification probability threshold of 0.9.

FIG. 6 shows a comparison of protocol performance across all conditions tested. The on-target methylation (left) and signal:background ratios (right) for all tested conditions are plotted, ranked from highest to lowest and colored by which enzyme was used. Hia5 outperformed EcoGII in nearly every setting tested. Pairwise comparisons of the two enzymes in the same conditions are included in the figures below.

FIG. 7 shows a comparison of permeabilization detergent across multiple replicates/conditions. Each “condition” (labeled arbitrarily with ‘A’, ‘B’, ‘C’, etc.) represents identical protocol conditions run on the same day, with the only variable being the detergent used. Thus, it is valid to compare bars grouped into the same condition, but not across conditions.

FIG. 8 shows comparisons of activation buffer/temperature conditions on DiMeLo-seq performance. Each “condition” (labeled arbitrarily with ‘A’, ‘B’, ‘C’, etc.) represents identical protocol conditions run on the same day, with the only variable being the methylation buffer and temperature used. Thus, it is valid to compare bars grouped into the same condition, but not across conditions.

FIG. 9 shows a comparison of three independent measurements of lamina association in chromosome 7 (FIG. 9A) and chromosome 3 (FIG. 9B) of HEK293T cells. Top row: Chromosome ideogram. Second row: Conventional DamID with short-read sequencing of DNA extracted from cells expressing Dam-LMNB1. Third row: mA/A counts in 100 kb bins from long-read nanopore sequencing and mA calling of DNA extracted from cells processed with DiMeLo-Seq targeting LMNB1. Fourth row: Long-read nanopore sequencing coverage across the entire chromosome from the third row data. Fifth row: mA/A counts in 100 kb bins from long-read nanopore sequencing and mA calling of DNA from nuclei processed using the DiMeLo-Seq protocol with no primary antibody. Sixth row: Coverage from short-read DamID-seq.

FIG. 10 shows DiMeLo-seq signal enrichment at CTCF peaks and at modified histone peaks. (FIG. 10A) Averaged CTCF-targeted methylation signal centered at published ChIP-seq peaks (from the same cell line, GM12878) and ranked in quartiles according to ChIP-seq peak signal strength (quartile 4, darkest line, shows the strongest signal in both the ChIP-seq and DiMeLo-seq datasets). Y axis indicates average mA probability score at each base position relative to the CTCF peak center. IgG isotype control shows that this targeting is specific. Untethered Hia5 shows that the oscillating pattern is due to increased DNA accessibility between highly phased nucleosomes surrounding CTCF. (FIG. 10B) Top: Averaged CTCF-targeted methylation signal centered at published ChIP-seq peaks. Bottom: CTCF-targeted methylation signal plotted as a heatmap, with each row corresponding to the region surrounding a single CTCF ChIP-seq peak in the genome, colored in 10-bp bins according to mA probability score. (FIG. 10C) As in B, but for different protein targets: the histone marks H3K9me3, H3K27me3, H3K9ac, H3K27ac.

FIG. 11 shows centromere enrichment by restriction digestion and size selection. (FIG. 11A) Illustration of the overall centromere enrichment strategy. (FIG. 11B) Simulated tradeoff between loss of sensitivity (% of alpha satellite lost) and gain of specificity (% of non-centromeric sequences not removed) as different size cutoff thresholds are used on genomic DNA digested with MscI+AseI (vertical line at 10 kb cutoff). (FIG. 11C) As in B but for gDNA digested with MscI only. Vertical line is shown at 50 kb. (FIG. 11D) Tapestation results illustrating the change in size distribution through the steps of the enrichment protocol. (FIG. 11E) Browser tracks illustrating the location of alpha satellite higher order repeat arrays (1st track); theoretical coverage assuming perfect size selection, perfect recovery, perfect mappability, and no sequencing bias (2nd track); actual coverage from DNA isolated by this strategy (3rd track); coverage from un-enriched gDNA sequencing (4th track). Overall, there is 20-fold higher coverage in centromere regions in the enriched track vs control track.

FIG. 12 shows simultaneous measurement of haplotype-specific protein-DNA interactions and CpG methylation. Phased reads are displayed across the IGF2/H19 Imprinting Control Region with CTCF sites indicated by triangles. Dots represent mA calls and squares represent mCpG calls. Heterozygous sites used for phasing are indicated with asterisks.

### DETAILED DESCRIPTION

The present disclosure provides methods and compositions to address the aforementioned unmet needs. For example, provided herein is a method for mapping specific protein-DNA interactions genome-wide, including highly repetitive areas of the genome, by performing targeted modifications of base-pairs at or near the genomic site where a protein of interest is interacting, followed by direct detection of those modified base-pairs using commercially available, long-read DNA sequencing platforms such as Oxford Nanopore Technologies' minION, or Pacific Biosciences HiFi sequencing. In some embodiments, the methods are referred to as “DiMeLo-Seq” which is short for Directed Methylation and Long-read Sequencing.

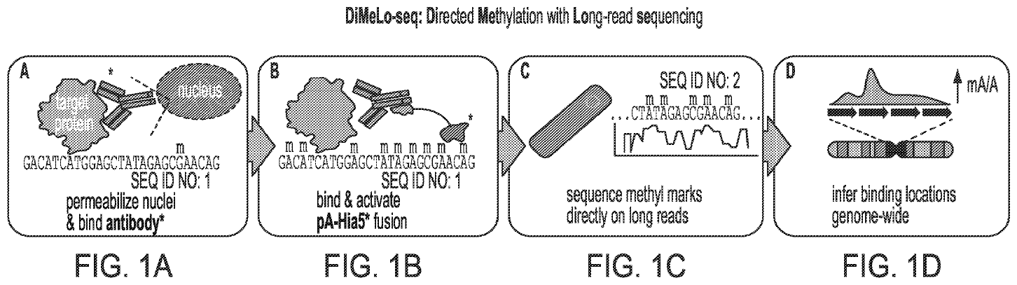

One exemplary embodiment of DiMeLo-Seq is explained below and shown in FIG. 1. By way of example, proteins of interest are targeted by a primary antibody in intact nuclei (FIG. 1A), as is common in other previous methods for measuring protein-DNA interactions with short-read sequencing technology such as ChIP-Seq (Barski, A., et al., (2007), Cell, 129(4), 823-837), CUT&RUN (Skene, P. J., & Henikoff, S. (2017), ELife, 6), and CUT&Tag (Kaya-Okur, H. S., et al., (2019), Nature Communications, 10(1), 1-10). Next, a methyltransferase, such as Dam, EcoGII, or Hia5 is fused to protein-A or protein-AG, and directed to the primary antibody and the protein of interest. Upon binding, the methyltransferase will methylate its target sequence (nearly any adenine in the case of EcoGII and Hia5) leaving a chemical recording of the binding or interacting sites on the DNA itself (FIG. 1B). Genomic DNA is then extracted and purified, and the base-modifications (i.e. mA) are directly detected on the native DNA molecules using long-read DNA sequencing (FIG. 1C). Long reads and the corresponding location of base-modifications are then mapped back to a complete assembly of the reference genome of the organism of interest (FIG. 1D). An accumulation of base-modifications along the genome will then correspond to the binding site, or interaction domain of the protein of interest, see FIG. 1.

As provided herein, DiMeLo-Seq is used, in various embodiments, to characterize binding sites and interacting domains of any protein that can be targeted with a primary antibody, including, for example, a nuclear lamina protein, a nucleolar protein, a transcription factor, a histone or histone variant, centromere protein A, an intracellular scFV, a chromatin-modifying enzyme, an RNA polymerase, a DNA polymerase, a DNA helicase, a DNA repair protein, a Cas9 protein, a dCas9 protein, a zinc finger protein, a TALE protein, a CTCF protein, a cohesion protein, a synaptonemal complex protein, a telomere-binding protein, a centromere-binding protein, and an outer kinetochore protein.

DiMeLo-Seq has key advantages over previous technologies including ChIP-Seq. CUT&RUN, CUT&TAG, ChIRP-Seq. Hi-C/4C, DamID, MadID, pA-DamID, and other techniques that profile the epigenetic state of cells using short-read sequencing technology. By implementing long-read sequencing to characterize interactions with the genome through targeted methylation, DiMeLo-Seq can map interactions in highly repetitive regions of the genome that cannot be mapped with short sequencing reads.

While DiMeLo-seq is useful for mapping protein-DNA interactions in repetitive regions, in other embodiments provided herein DiMeLo-seq also provides additional single-molecule information that can be leveraged in several ways. For example, in one embodiment endogenous CpG methylation can be jointly measured along with protein-DNA interaction sites on the same single molecules of DNA. This is useful when studying how DNA methylation and protein binding interact, for example when DNA methylation abolishes the binding of certain transcription factors. Additionally, because methyltransferases favor accessible linker DNA between nucleosomes, nucleosome positioning can be inferred based on the density of methylation marks, as with existing long-read accessibility measurement technologies (Shipony, Z., et al., (2020), Nature Methods, 17(3), 319-327; Stergachis, A. B., et al., (2020), Science, 368(6498), 1449-1454; Lee, I., et al., (2020), Nature Methods, 17(12), 1191-1199; Wang. Y., et al., (2019), Genome Research, 29(8), 1329-1342; and Abdulhay, N., et al., (2020), eLife, 9, e59404.). Because it is an amplification-free method, it also allows one to linearly infer the frequency of binding of a protein at a particular site in a population of cells. Furthermore, since the enzyme reach is on the order of 100-200 bp, and reads can regularly be as long as hundreds of kb, we can infer multiple protein-DNA binding events on single molecules. This is useful for exploring the density of a protein along a stretch of chromatin, or the exact joint binding profile of proteins to proximal sites. It also lends itself to examining the joint distribution of multiple proteins, each fused to a distinct DNA-modifying enzyme, on the same long single molecule of DNA.

Additionally, in still other embodiments DiMeLo-Seq could be implemented to target long non-coding RNAs to profile their regulatory interactions with chromatin, similar to techniques such as ChIRP-seq (Chu, C., et al., (2011), Mol Cell, 44, 667-678). DiMeLo-Seq could also be used to target dCas9 or similar proteins to probe topologically interacting domains between genomic loci or three-dimensional chromatin organization in a fashion similar to chromatin conformation capture techniques (Kempfer, R. & Pombo, A., (2020), Nat Rev Genet, 21, 207-226).

Measuring protein-DNA interactions has been described in, for example, Van Steensel, et al., (2001), Nature Genetics, 27(3), 304-308; Vogel, M. J., et al., (2007), Nature protocols, 2(6), 1467-1478; Wu, F., et al., (2016), Journal of Visualized Experiments, 2016(107), 53620; Michal Sobecki, et al., (2018), Cell Reports, 25(10), 2891-2903.e5.; Kind. J., et al., (2015), Cell, 163(1), 134-147; Altemose, N., et al., (2020), Cell Systems, 11(4), 354-366.e9; Skene, P. J., & Henikoff, S. (2017), ELife, 6; Meers, M. P., et al., ELife, 8; Kaya-Okur, H. S., et al., (2019), Nature Communications, 10(1), 1-10; Robertson, G., et al., (2007), Nature methods, 4(8), 651-657; and van Schaik, T., et al., (2020), EMBO reports, 21(11), e50636.

Measuring RNA-DNA interactions has been described in, for example, Chu, C., et al., (2011), Molecular Cell, 44(4), 667-678; and Cheetham, S. W., et al., Nat Struct Mol Biol 25, 109-114 (2018). Measuring DNA-DNA interactions has been described in, for example, Simonis, M., et al., (2006), Nature Genetics, 38(11), 1348-1354; Lieberman-Aiden, E., et al., (2009), Science, 326(5950), 289-293; Krijger, P. H. L., et al., (2020), Methods, 170, 17-32. Long-read detection of targeted methylation (chromatin accessibility) has been discussed previously in, for example, Shipony, Z., et al., (2020), Nature Methods, 17(3), 319-327; Stergachis, A. B., et al., (2020), Science, 368(6498), 1449-1454; Lec, I., et al., (2020), Nature Methods, 17(12), 1191-1199; Wang. Y., et al., (2019), Genome Research, 29(8), 1329-1342; and Abdulhay, N., et al., (2020), eLife, 9, c59404.

As described herein, numerous enrichments steps and procedures are provided to preserve long DNA fragments for use the methods of present disclosure. In some embodiments, samples are enriched for binding to the protein of interest by first performing immunoprecipitation of the sample chromatin with an antibody targeting the protein, while preserving long DNA fragments. Alternatively, one could perform immunoprecipitation on the purified DNA itself using an antibody targeting, for example, m6A. In still other embodiments, one could also enrich for large fragments of m6A-containing DNA by digesting the isolated genomic DNA with a methyl-sensitive restriction enzyme like DpnII, which only cuts unmethylated GATC sites, then remove small digestion products by standard DNA size selection methods. In other embodiments, one could also enrich for a particular region of the genome, without enriching for the protein of interest or the m6A mark specifically, by using existing amplification-free targeted long-read sequencing approaches (e.g. Read-Until, UNCALLED, or Cas9-targeted adapter insertion). In still other embodiments, if one is targeting repetitive regions of the genome, one could use a method provided herein which entails digesting the genome with restriction enzymes that tend to cut outside the targeted repetitive region, but not inside it, followed by removal of small fragments by standard DNA size selection methods.

### Definitions

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 109 nucleotides or larger. Polynucleotides and nucleic acids include RNA, cDNA, genomic DNA. In particular, the polynucleotides and nucleic acids of the present invention refer to polynucleotides encoding a chromatin protein, a nucleotide modifying enzyme and/or fusion polypeptides of a chromatin protein and a nucleotide modifying enzyme, including mRNAs, DNAs, cDNAs, genomic DNA, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and more preferably 80%, still more preferably 90% sequence identity, to a native chromatin binding protein or to a nucleotide modifying enzyme.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded or double-stranded.

The term “protein” or “protein of interest” refers to a polymer of amino acid residues, wherein a protein may be a single molecule or may be a multi-molecular complex. The term, as used herein, can refer to a subunit in a multi-molecular complex, polypeptides, peptides, oligopeptides, of any size, structure, or function. It is generally understood that a peptide can be 2 to 100 amino acids in length, whereas a polypeptide can be more than 100 amino acids in length. A protein may also be a fragment of a naturally occurring protein or peptide. The term protein may also apply to amino acid polymers in which one or more amino acid residues is an artificial chemical analogue of a corresponding naturally occurring amino acid. A protein can be wild-type, recombinant, naturally occurring, or synthetic and may constitute all or part of a naturally-occurring, or non-naturally occurring polypeptide. The subunits and the protein of the protein complex can be the same or different. A protein can also be functional or non-functional.

Non-limiting examples of a biomolecule of interest, e.g., a protein or protein of interest include, without limitation, a nuclear lamina protein (e.g., LMNB1 and LMNA), a nucleolar protein (e.g., NPM1 and NCL), a transcription factor (e.g., NPAT and SOX9), a histone or histone variant (e.g., centromere protein A (CENPA) and H3K9ac), centromere protein A, a modification-specific internal antibody (mintbody) (e.g., H3K9ac mintbody and H4K20me1 mintbody), an intracellular scFV, a nanobody, a chromatin-modifying enzyme (e.g., PRDM9 and HDAC2), an RNA polymerase subunit or modifier (e.g., RPB1 and CDK9), a DNA polymerase subunit or modifier (e.g., POLB and POLA2), a DNA helicase (e.g., MCM2 and RECQ1), a DNA repair protein (e.g., RAD51 and FANCD2), a Cas9 protein, a dCas9 protein, a zinc finger protein (e.g., PRDM9 and ZNF212), an engineered TALE protein, a CTCF protein, a cohesion protein (e.g., RAD21 and SMC1A), a synaptonemal complex protein (e.g., SYCP1 and SYCP2), a telomere-binding protein (e.g., TRF1 and TRF2), a centromere-binding protein (e.g., CENPC and CENPT), and an outer kinetochore protein (e.g., SPC24 and SPC25). Additional proteins are described herein.

The term “chromatin” as used herein refers to a complex of DNA and protein, both in vitro and in vivo. This includes all proteins that are directly contacting DNA, and also proteins that are part of a protein or ribonucleoprotein complex that may be associated with DNA. A chromatin protein may or may not directly contact DNA. Chromatin also includes proteins that are transiently associated with DNA, with DNA-protein, or with DNA-ribonucleoprotein complexes, i.e., only during part of the cell cycle. “Chromatin protein” includes, but is not limited to histones, transcriptional factors, centromere proteins, heterochromatin proteins, cuchromatin proteins, condensins, cohesins, origin recognition complexes, histone kinases, dephosphorylases, acetyltransferases, deacetylases, methyltransferases, demethylases, and other enzymes that covalently modify histone, DNA repair proteins, proteins involved in DNA replication, proteins involved in transcription, proteins part of dosage compensation complexes and X-chromosome inactivation, proteins that are part of chromatin remodeling complexes, telomeric proteins, and the like.

The term “polypeptide” refers to a polymer of amino acids and its equivalent and does not refer to a specific length of the product; thus, peptides, oligopeptides and proteins are included within the definition of a polypeptide. A “fragment” refers to a portion of a polypeptide having typically at least 10 contiguous amino acids, more typically at least 20, still more typically at least 50 contiguous amino acids of the protein. A “derivative” is a polypeptide which is identical or shares a defined percent identity with the wild-type protein or nucleotide modification enzyme. The derivative can have conservative amino acid substitutions, as compared with another sequence. Derivatives further include, for example, glycosylations, acetylations, phosphorylations, and the like. Further included within the definition of “polypeptide” are, for example, polypeptides containing one or more analogs of an amino acid (e.g., unnatural amino acids, and the like), polypeptides with substituted linkages as well as other modifications known in the art, both naturally and non-naturally occurring. Ordinarily, such polypeptides will be at least about 50% identical to the native protein or nucleotide modification enzyme acid sequence, typically in excess of about 90%, and more typically at least about 95% identical. The polypeptide can also be substantially identical as long as the fragment, derivative or analog displays similar functional activity and specificity as the wild-type protein or nucleotide modification enzyme.

The terms “amino acid” or “amino acid residue”, as used herein, refer to naturally occurring L amino acids or to D amino acids as described further below. The commonly used one- and three-letter abbreviations for amino acids are used herein (see, e.g., Alberts et al. Molecular Biology of the Cell, Garland Publishing, Inc., New York (3d ed. 1994)).

The term “isolated” refers to a nucleic acid or polypeptide that has been removed from its natural cellular environment. An isolated nucleic acid is typically at least partially purified from other cellular nucleic acids, polypeptides and other constituents.

“Functionally active polypeptide” or “biologically active fragments” refers to those fragments, derivatives and analogs displaying the functional activities associated with a full length protein of interest.

The terms “identical” or “percent identity.” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection.

The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

“Similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 30%, 40%, 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below. The term “substantial similarity” in the context of polypeptide sequences, indicates that the polypeptide comprises a sequence with at least 70% sequence identity to a reference sequence, or preferably 80%, or more preferably 85% sequence identity to the reference sequence, or most preferably 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ only by one or more conservative substitutions.

The term “conservative substitution,” when describing a polypeptide, refers to a change in the amino acid composition of the polypeptide that does not substantially alter the polypeptide's activity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, and the like) such that the substitution of even critical amino acids does not substantially alter activity. Conservative substitution tables providing functionally similar amino acids are well known in the art. For example, the following six groups each contain amino acids that are conservative substitutions for one another: 1) alanine (A), serine (S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.” For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, for example, by the local homology algorithm of Smith & Waterman (Adv. Appl. Math. 2:482 (1981), which is incorporated by reference herein), by the homology alignment algorithm of Needleman & Wunsch (J. Mol. Biol. 48:443-53 (1970), which is incorporated by reference herein), by the search for similarity method of Pearson & Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, WI), or by visual inspection. (See generally Ausubel et al. (eds.), Current Protocols in Molecular Biology, John Wiley and Sons, New York (1996)).

One example of a useful algorithm is PILEUP. PILEUP creates a multiple sequence alignment from a group of related sequences using progressive, pairwise alignments to show the percent sequence identity. It also plots a tree or dendrogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins & Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al, Nucleic Acid Res. 26:3986-90 (1998); Altschul et al, Nucleic Acid Res. 25:3389-402 (1997), which are incorporated by reference herein). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915-9 (1992), which is incorporated by reference herein) alignments (B) of 50, expectation (E) of 10. M=5, N=−4, and a comparison of both strands.

In addition to calculating percent sequence identity, the BLAST algorithm also performs a statistical analysis of the similarity between two sequences (see, e.g., Karlin & Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001. Further, a polypeptide is typically substantially identical to a second polypeptide, for example, where the two peptides differ only by conservative substitutions. The terms “transformation” or “transfection” means a process of stably or transiently altering the genotype of a recipient cell or microorganism by the introduction of polynucleotides. This is typically detected by a change in the phenotype of the recipient cell or organism. The term “transformation” is generally applied to microorganisms, while “transfection” is used to describe this process in cells derived from multicellular organisms.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al. Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

Polypeptide derivatives include naturally-occurring amino acid sequence variants as well as those altered by substitution, addition or deletion of one or more amino acid residues that provide for functionally active molecules. Polypeptide derivatives include, but are not limited to, those containing as a primary amino acid sequence all or part of the amino acid sequence of a native protein of interest or chromatin polypeptide including altered sequences in which one or more functionally equivalent amino acid residues (e.g., a conservative substitution) are substituted for residues within the sequence, resulting in a silent change.

In another aspect, polypeptides of the present invention include those peptides having one or more consensus amino acid sequences shared by all members of the protein of interest, but not found in other proteins. Database analysis indicates that these consensus sequences are not found in other polypeptides, and therefore this evolutionary conservation reflects the nucleotide target binding-specific function of the protein of interest or chromatin polypeptides. Polypeptide family members, including fragments, derivatives and/or analogs comprising one or more of these consensus sequences, are also within the scope of the present disclosure.

In another embodiment, a polypeptide consisting of or comprising a fragment of a protein of interest or chromatin polypeptide having at least 5 contiguous amino acids of the protein of interest which recognize the specific target nucleotide sequence is provided. In other embodiments, the fragment consists of at least 20 or 50 contiguous amino acids of the protein of interest or chromatin polypeptide. In a specific embodiment, the fragments are not larger than 35, 100 or even 200 amino acids. Fragments, derivatives or analogs of chromatin polypeptide include, but are not limited to, those molecules comprising regions that are substantially similar to a chromatin polypeptide or fragments thereof (e.g., in various embodiments, at least 30%, 40%, 50%, 60%, 70%, 75%, 80%, 90%, or even 95% identity or similarity over an amino acid sequence of identical size), or when compared to an aligned sequence in which the alignment is done by a computer sequence comparison/alignment program known in the art, as described above, or whose coding nucleic acid is capable of hybridizing to a nucleic acid sequence encoding a protein of interest or chromatin protein, under high stringency, moderate stringency, or low stringency conditions. The choice of hybridization conditions will generally be guided by the purpose of the hybridization, the type of hybridization (DNA-DNA or DNA-RNA), and the level of relatedness between the sequences. Methods for hybridization are well established in the literature; See, for example: Sambrook, supra.; Hames and Higgins, eds, Nucleic Acid Hybridization A Practical Approach, IRL Press, Washington DC, (1985); Berger and Kimmel, eds, Methods in Enzymology, Vol. 52, Guide to Molecular Cloning Techniques, Academic Press Inc., New York, NY, (1987); and Bothwell et al, eds, Methods for Cloning and Analysis of Eukaryotic Genes, Jones and Bartlett Publishers, Boston, M A (1990); which are incorporated by reference herein in their entirety. The stability of nucleic acid duplexes will decrease with an increased number and location of mismatched bases; thus, the stringency of hybridization may be used to maximize or minimize the stability of such duplexes. Hybridization stringency can be altered by: adjusting the temperature of hybridization; adjusting the percentage of helix-destabilizing agents, such as formamide, in the hybridization mix; and adjusting the temperature and salt concentration of the wash solutions. In general, the stringency of hybridization is adjusted during the post-hybridization washes by varying the salt concentration and/or the temperature. Stringency of hybridization may be reduced by reducing the percentage of formamide in the hybridization solution or by decreasing the temperature of the wash solution. High stringency conditions involve high temperature hybridization (e.g., 65-68° C. in aqueous solution containing 4 to 6×SSC, or 42° C. in 50% formamide) combined with washes at high temperature (e.g., 5 to 25° C. below the Tₘ) at a low salt concentration (e.g., 0.1×SSC). Reduced stringency conditions involve lower hybridization temperatures (e.g., 35-42° C. in 20-50% formamide) with washes at intermediate temperature (e.g., 40 to 60° C.) and in a higher salt concentration (e.g., 2 to 6×SSC). Moderate stringency conditions involve hybridization at a temperature between 50° C. and 55° C. and washes in 0.1×SSC, 0.1% SDS at between 50° C. and 55° C.

Nucleotide modifying enzymes (e.g., “enzyme capable of modifying genomic DNA”), fragments, derivatives and analogs thereof useful in the present invention are those which can modify one or more nucleotides in a nucleic acid sequence, such as an RNA, DNA, or the like, under conditions found in vitro or in situ or in a live cell and in a manner which is detectable. The enzyme, in some embodiments, will optionally modify the nucleotides in a manner which is not toxic to the cell. In other words, the cell or organism must be able to continue to proliferate and differentiate in a normal manner. For the modification to be detectable, an enzyme is selected which modifies the nucleotide in a manner which is not typical of a modification commonly found in the cell being assayed. For instance, in eukaryotic cells it is typical to select as the modification enzyme, for example, DNA adenine methyl transferase because methylation of adenine is not common in eukaryotic cells. Additional nucleotide modification enzymes useful in the present invention include, for example, but are not limited to, adenine methyltransferases, cytosine methyltransferases, thymidine hydroxylases, hydroxymethyluracil β-glucosyl transferases, adenosine deaminases, and the like. In other embodiments, the enzyme capabale of modifying genomic DNA includes ten-eleven translocation (TET) dioxygenase (e.g., enzymes that generate 5-hydroxymethylcytosine, 5-carboxylcytosine, 5-formylcytosine). In still another embodiment, the use of a methyltransferase with modified SAM is provided to deposit a different mark rather than a methyl group. Optionally this would still involve using a (potentially modified or unmodified) methyltransferase but it would not be a methyl group being deposited. In still other embodiments, the enzyme is miniSOG or SOPP2 which, when excited with blue light, generate highly reactive singlet oxygen molecules, which oxidize guanines in their vicinity.

The present disclosure provides methods and materials for determining the genomic location of at least one biomolecule-genomic DNA interaction. In one embodiment, the method comprises one or more or all of the following steps, (a) incubating a biomolecule of interest under conditions that allow the biomolecule of interest to contact a genomic DNA sequence; (b) isolating and permeabilizing nuclei from the cells in (a) under conditions that allow isolation of genomic DNA bound by the biomolecule of interest; (c) contacting the biomolecule bound to genomic DNA with a first binding moiety capable of specifically binding to the biomolecule of interest; (d) contacting the first binding moiety with a second binding moiety capable of specifically binding to the first binding moiety, wherein said second binding moiety is conjugated to an enzyme capable of modifying genomic DNA; (c) incubating the first binding moiety and second binding moiety of (d) under conditions that allow modification of genomic DNA; (f) isolating and preparing the genomic DNA for sequencing, wherein said preparing does not require amplification of the DNA; and (g) sequencing the genomic DNA under conditions that allow determining the location of the biomolecule-DNA interaction.

The “conditions” for the aforementioned steps are described herein. With respect to permeabilizing nuclei, the conditions should be such that nuclei are sufficiently permeabilized for binding moieties and modifying enzymes to diffuse in, while minimally disrupting nuclear chromatin structure or shearing DNA, and while allowing nuclei to be isolated (by centrifugation or magnetic beads) to facilitate buffer exchange throughout the protocol as described herein. In some embodiments, nuclei are permeabilize with 0.02% digitonin for 5 minutes on ice with 20 mM HEPES-KOH, 150 mM NaCl, 0.5 mM Spermidine, 0.1% BSA, one Roche Complete tablet—EDTA. In other embodiments, all remaining washes include 0.1% Tween-20 and do not include digitonin. In still other embodiments, the incubating conditions can include or exclude EDTA, salt, BSA, at various times and temperatures as described herein. In one embodiment, for activation, incubation with 15 mM Tris, pH 8.0, 15 mM NaCl, 60 mM KCl, 1 mM EDTA, pH 8.0, 0.5 mM EGTA. pH 8.0, 0.5 mM Spermidine, 0.1% BSA, 800 uM SAM is contemplated. Incubation at 37 C for pA-Hia5, or 30 C for pAG-EcoGII, are also contemplated. In some embodiments, low salt and BSA are contemplated and provided herein. In still other embodiments, the “preparing the DNA for sequencing” step comprises Ampure/SPRI beads, PCI extraction, spin column extraction, and/or spooling.

In various embodiments, “a first binding moiety capable of specifically binding to the biomolecule of interest.” is an antibody or antibody fragment capable of binding to the biomolecule of interest.

In various embodiments, “a second binding moiety capable of specifically binding to the first binding moiety, wherein said second binding moiety is conjugated to an enzyme capable of modifying genomic DNA” is an antibody, protein-A, protein-G, protein-A/G.

As used herein, “contacted” or “interaction” or “interaction site” as it relates to protein-DNA interactions includes direct contact or binding of a protein to a DNA at, for example, a DNA-binding site or sequence, and further includes indirect contact whereby a protein comes in sufficiently close proximity to a DNA sequence that allows a “mark” or other change to be imparted on the DNA sequence, as described herein. In other embodiments, nucleotides can be modified (i.e., marked) wherein the nucleotides are not near the interaction site or point of contact, but rather are topologically near the DNA binding site of the biomolecule. The phrase “topologically near the genomic DNA binding site of the biomolecule” as used herein thus refers to nucleotides that are brought near a DNA interaction site by virtue of three-dimensional or conformational properties.

In some embodiments, the contact or interaction between a DNA and a protein occurs in vivo (e.g., inside a cell). In other embodiments, the interaction occurs in vitro. In some embodiments, measurements are made both in vivo and in situ (e.g., multiplexing). For example, a DNA-modifying enzyme can, in one embodiment, be recruited to one target in vivo and then a different DNA-modifying enzyme can be recruited to another target in situ. The present disclosure thus contemplates performing a DamID protocol, in which a cell is engineered to express a protein of interested fused to the methyltransferase to map interactions in vivo with short read sequencing, in combination with a DimeLo-seq on such an engineered cell to measure two protein-DNA interactions at once.

In some embodiments, for example where multiple DNA-protein interactions are determined or measured, multiple (different) DNA-modifying proteins (e.g., enzymes) can be used.

In various embodiments, short-read (e.g., 200-300 bp) and long-read sequencing methods may be used with the methods provided herein, including using DamID protocols.

In still other embodiments, the methods provided herein allow for sequencing long stretches of DNA. For example, in various embodiments, 500, 1,000, or 10,000 bp or more may be sequenced.

In various embodiments, the protein of interest is a native protein, a wild-type protein, or a recombinant protein. In some embodiments, the protein is naturally-expressed by the cell or the cell is engineered to express, e.g., a recombinant protein, under specific conditions.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

The following materials and methods were used in in the Examples described herein.

### Example 1

### Exemplary DimeLo-Seq Protocol

This Example provides exemplary materials and methods for DimeLo-seq.

### I. Perform In Situ Targeted Methylation and DNA Extraction

### A. Reagent Preparation:

Prepare all reagents fresh and keep on ice. Syringe filter all solutions through a 0.2 uM filter.
1. 5% digitonin solution: Solubilize digitonin in preheated 95° C. Milli-Q water to create a 5% digitonin solution (e.g. 10 mg/200 μl).
2. Wash buffer:

| | | |
|---|---|---|
| component | amount | concentration |

| | | | | |
|---|---|---|---|---|
| HEPES-KOH, 1M, pH 7.5 | 1 | ml | 20 | mM |
| NaCl, 5M | 1.5 | ml | 150 | mM |
| Spermidine, 6.4M | 3.91 | μl | 0.5 | mM |

| | | | |
|---|---|---|---|
| Roche Complete tablet -EDTA | 1 | tablet | — |
| BSA | 50 | mg | 0.1% |

| | | |
|---|---|---|
| H2O | up to 50 ml | — |

3. Dig-Wash buffer: Add 0.02% digitonin to wash buffer. For example, add 20 μl of 5% digitonin solution to 5 ml wash buffer.
4. Tween-Wash buffer: Add 0.1% Tween-20 to wash buffer. For example, add 50 μl Tween-20 to 50 ml wash buffer.
5. Activation buffer: Create the activation buffer but wait to add SAM until the activation step.

| component | amount | | concentration | |
|---|---|---|---|---|
| Tris, pH 8.0 1M | 750 | uL | 15 | mM |
| NaCl 5M | 150 | uL | 15 | mM |
| KCl 1M | 3 | mL | 60 | mM |
| EDTA, pH 8.0 0.5M | 100 | uL | 1 | mM |
| EGTA, pH 8.0 0.5M | 50 | uL | 0.5 | mM |
| Spermidine, 6.4M | 3.91 | uL | 0.5 | mM |

| | | | |
|---|---|---|---|
| BSA | 50 | mg | 0.1% |

| H2O | up to 50 mL | — | |
|---|---|---|---|
| SAM, 32 mM | 2.5 ul per 100 ul activation | 800 | μM |
| | reaction (add at activation step) | | |

### B. Protocol

All spins are at 4° C. for 3 minutes at 500×g. To prevent nuclei from lining the side of the tube, break all spins into two parts: 2 minutes with the tube hinge facing inward, followed by 1 minute with the tube hinge facing outward. Use wide bore tips when working with nuclei. In certain embodiments, do not use Triton (0.1%) or NP-40. Both reduce methylation activity.

Optimal digitonin concentration may vary by cell type. For HEK293T, GM12878, HG002, and Hap1 cells, 0.02% works well. One can test different concentrations of digitonin and verify permeabilization and nuclear integrity by Trypan blue staining. Thus, in some embodiments, 0.02% to 0.1% digitonin is contemplated herein. Tween is used to reduce hydrophilic non-specific interactions and BSA to reduce hydrophobic non-specific interactions. In some embodiments, use of BSA at the activation step significantly increases methylation activity.

Optimal primary antibody concentration may vary by protein target of interest. A 1:50 dilution (e.g., approx. 20 ug/ml) works well for targeting LMNB1 and is likely a good starting point for most antibodies. A secondary antibody binding step following primary antibody binding and before, for example, pA-Hia5 binding can reduce total methylation and specificity. Including a secondary antibody binding step is, in one embodiment, not performed.

In some embodiments of DimeLo-seq, a fixation method is performed as follows
1. Resuspend cells in PBS.
2. Add PFA to 0.1% (e.g. 6.2 μl of 16% PFA to 1 ml cells) for 2 minutes while gently vortexing.
3. Add 1.25 M glycine (sterile; 0.938 g in 10 ml) to twice the molar concentration of PFA to stop the crosslinking (e.g. 60 μl of 1.25 M glycine to 1 ml)
4. Centrifuge 3 minutes at 500×g at 4° C. and remove the supernatant
5. Resuspend the fixed cells in Dig-Wash buffer (A. Nuclear isolation, step 3).

### A. Nuclear Isolation

1. Prepare cells (1 M-5 M per condition)
2. Wash cells in PBS. Spin and remove supernatant.
3. Resuspend cells in 1 ml Dig-Wash buffer. Incubate for 5 minutes on ice.
4. Split nuclei suspension into separate tubes for each condition.
5. Spin and remove supernatant.

### B. Primary Antibody Binding

1. Gently resolve each pellet in 200 μl Tween-Wash containing primary antibody at 1:50 (e.g., 20 ug/ml).
2. Place on rotator at 4° C. for ˜2 hr.
3. Spin and remove supernatant.
4. Wash twice with 0.95 ml Tween-Wash. For each wash, gently and completely resolve the pellet. This may take pipetting up and down ˜10 times. Following resuspension, place on rotator at 4° C. for 5 minutes before spinning down.
   C. pA-Hia5 Binding
1. Gently resolve pellet in 200 μl Tween-Wash containing 200 nM pA-Hia5. See protein quantification protocol below.
2. Place on rotator at room temperature for ˜1 hr.
3. Spin and remove supernatant.
4. Wash with 0.95 ml Tween-Wash. For each wash, gently and completely resolve the pellet. Following resuspension, place on rotator at 4° C. for 5 minutes before spinning down.

### Protein Quantification Protocol:

1. Thaw protein from −80° C. at room temperature and then move to ice immediately.
2. Spin at 4° C. for 10 minutes at 10,000×g or higher.
3. Transfer supernatant to a new tube.
4. Use Qubit with 2 μl sample volume to quantify protein.

### D. Activation

1. Gently resolve pellet in 100 μl of Activation Buffer per sample. Be sure to add SAM to 800 uM to the activation buffer at this step!
2. Incubate at 37° C. for 30 minutes.
3. Spin and remove supernatant.
4. Resuspend in 100 μl cold PBS.

### E. DNA Extraction

In some embodiment, a kit such as the Monarch Genomic DNA Purification Kit is used.

### II. Perform Library Preparation and Start the Sequencing Run

In some embodiments of DimeLo-seq, the Nanopore protocol for Native Barcoding Ligation Kit is used with the following modifications:
1. Load ˜3 μg DNA into end repair.
2. Incubate for 10 minutes at 20° C. for end repair instead of 5 minutes.
3. Load ˜1 μg of end repaired DNA into barcode ligation.
4. Double the ligation incubation time to at least 20 minutes.
5. Elute in 18 μl instead of 26 μl following barcode ligation reaction cleanup to allow for more material to be loaded into the final ligation.
6. Load ˜3 μg of pooled barcoded material into the final ligation. If needed, concentrate using speedvac to be able to load 3 μg into the final ligation.
7. Double the ligation incubation time to at least 20 minutes.
8. Use Long Fragment Buffer (LFB) (not ethanol) for the final cleanup.
9. Perform final elution in 13 μl EB. Take out 1 μl to dilute 1:5 for quantification by Qubit (and size distribution analysis by TapeStation/Bioanalyzer if desired).
10. Load ˜500 ng-1 μg of DNA onto the sequencer.
11. Bubbles will destroy pores and ruin runs; mix and spin down all flush/wash solutions really well to eliminate bubbles.
12. The Flow Cell Wash Kit can increase the throughput per flowcell with <1% carryover of pre-wash barcodes.
13. Spiking in more library+SQB+LB during a run, without a wash step, can also increase pore occupancy if it's low.

### Example 2

### Assessing Methylation Efficiency In Vitro

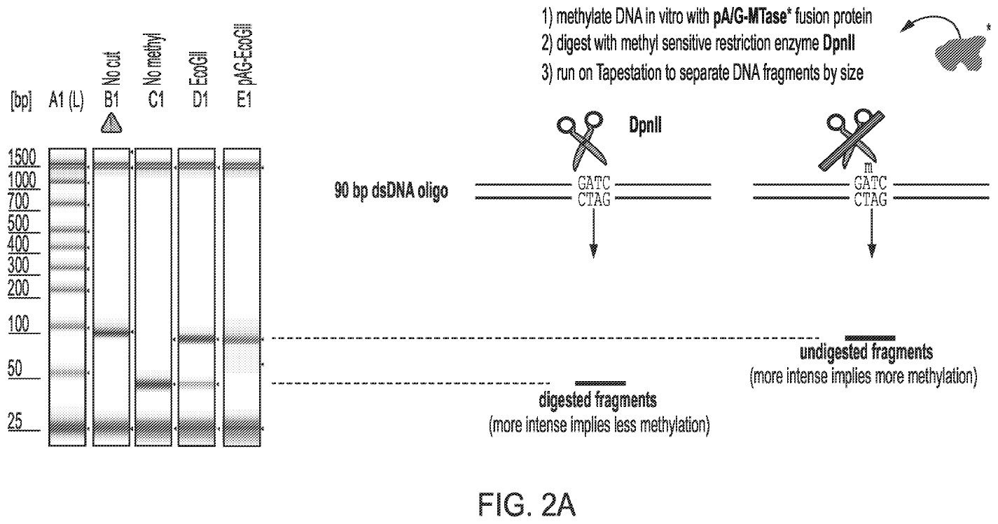

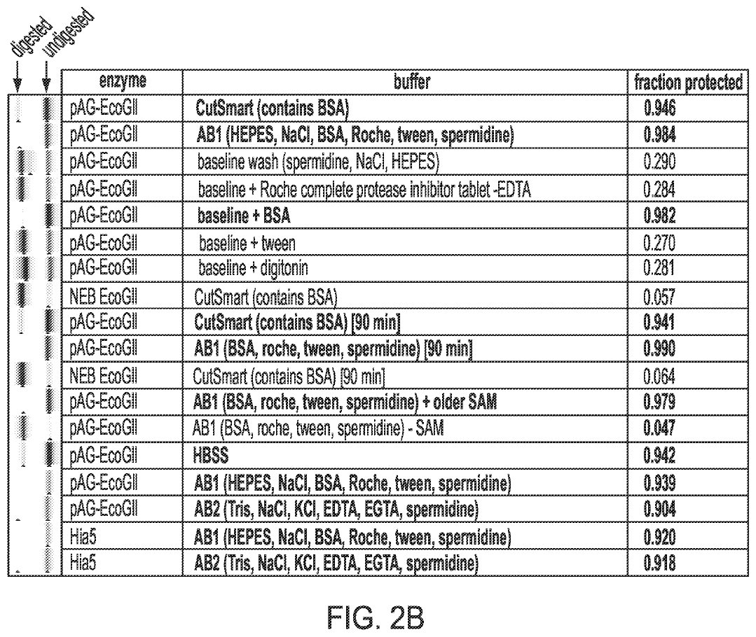

An in vitro digestion assay was used to test the methylation efficiency of the methyltransferases and methyltransferase-protein A/G fusion proteins in a variety of buffers. The methyltransferase was incubated with a 90 bp synthetic oligonucleotide with a single GATC motif, the restriction site for DpnII, near its center (FIG. 2). Methylation at the GATC motif protects this oligo from DpnII digestion. Therefore, fragment size analysis after DpnII digestion serves as a proxy for methylation efficiency of the pAG-EcoGII.

This analysis showed that these adenine methyltransferases and their pA/G fusions are active in a wide range of buffers (composition of activation buffers AB1 and AB2 are listed below). The addition of 0.1% BSA improves in vitro methylation efficiency. Hia5 and EcoGII performance were compared with various buffers with this in vitro digestion assay and were indistinguishable, with both showing close to 100% methylation efficiency.

Activation Buffer 1 AB1

| | | |
|---|---|---|
| component | amount | concentration |

| | | | | |
|---|---|---|---|---|
| HEPES-KOH, 1M, pH 7.5 | 1 | ml | 20 | mM |
| NaCl, 5M | 1.5 | ml | 150 | mM |
| Spermidine, 6.4M | 3.91 | μl | 0.5 | mM |

| | | | |
|---|---|---|---|
| Roche Complete tablet -EDTA | 1 | tablet | — |
| BSA | 50 | mg | 0.1% |

| | | |
|---|---|---|
| H2O | up to 50 ml | — |

Activation Buffer 2 (AB2)

| | | |
|---|---|---|
| component | amount | concentration |

| | | | | |
|---|---|---|---|---|
| Tris, pH 8.0 1M | 750 | uL | 15 | mM |
| NaCl 5M | 150 | uL | 15 | mM |
| KCl 1M | 3 | mL | 60 | mM |
| EDTA, pH 8.0 0.5M | 100 | uL | 1 | mM |
| EGTA, pH 8.0 0.5M | 50 | uL | 0.5 | mM |
| Spermidine, 6.4M | 3.91 | uL | 0.5 | mM |

| | | | |
|---|---|---|---|
| BSA | 50 | mg | 0.1% |

| H2O | up to 50 mL | — | |
|---|---|---|---|
| SAM, 32 mM | (add at activation step) | 800 | μM |

### Example 3

Profiling Lamina-Associated Domains with DiMeLo-Seq

Large regions of the genome are known to be regularly associated with the nuclear lamina. These lamina-associated domains (LADs) have been well characterized in drosophila (Pickersgill, H., et al., (2006). Nat Genet, 38, 1005-1014) and mammalian cells (Guelen, L., et al., (2008), Nature, 453, 948-951) using DNA Adenine Methyltransferase Identification (DamID), a technique which also uses an adenine methyltransferase (Dam) to record protein-DNA interactions. However, DamID instead reads out methyladenines by 1) digesting the genome with the methyladenine-specific restriction enzyme DpnI, 2) amplifying short (<500 bp) fragments produced by the digestion, and 3) sequencing the ends of those short, amplified DNA fragments using short, high-throughput sequencing reads (Wu, F., et al., (2016), J. Vis. Exp., 107, c53620).

Due to the lack of GATC sites in many repetitive regions (Sobecki, M., et al., (2018), Cell Reports, 25, 2891-2903), even with complete genome assemblies, these GATC-poor repetitive regions cannot be probed by conventional DamID. Other protein-DNA mapping approaches, such as MadID, ChIP-seq, and CUT&RUN, can recover information from these repetitive regions, but they produce short sequencing reads that cannot be mapped unambiguously within highly repeated sequences. This underlines the need for a technology like DiMeLo-seq that is able to map protein-DNA interactions using long sequencing reads, which can map to and cover repetitive regions more comprehensively.

In previous studies, LADs have been mapped by short-read DamID in numerous cell lines by engineering cells to express in vivo a fusion complex between Dam and Lamin B1 (LMNB1), a nuclear lamina protein (van Steensel, B. & Belmont, A. S., (2017), Cell, 169, 780-791). Some regions of the genome are known to be in contact with the nuclear lamina in every cell type studied (called constitutive LADs, or cLADs), and some are known to never contact the nuclear lamina in any cell types studied (called constitutive inter-LADs, or ciLADs). Recently, cLADs and ciLADs in HEK293T cells in both bulk samples and single cells were characterized (Altemose, N., et al., (2020), Cell Systems, 11(4), 354-366). These regions serve as useful positive and negative controls, allowing us to determine the amount of on-target and off-target methylation (and the ratio between these) while optimizing DiMeLo-seq. Furthermore, because LMNB1 binds large genomic regions (median length 500 kb), we can evaluate the performance of DiMeLo-seq using low-coverage sequencing data. Finally, LMNB1 occupies a very distinct space in the nucleus, allowing us to use immunofluorescence experiments to validate that our antibodies and methyltransferase constructs were targeting the nuclear lamina efficiently and specifically. Thus, LMNB1 served as an ideal target for initial testing and optimization of the DiMeLo-seq method.

### Assessing Targeted Methylation Efficiency In Situ Prior to Sequencing

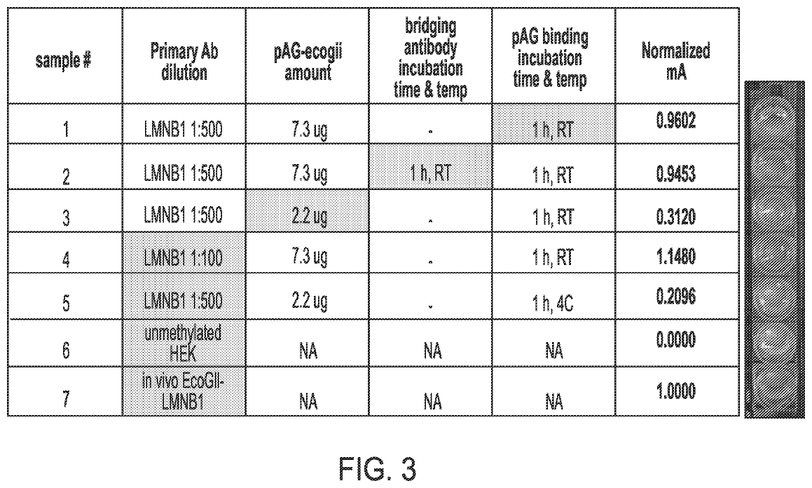

Targeted methylation of lamina associated genomic DNA in nuclei was initially optimized by performing the DiMeLo-seq protocol on extracted HEK293T nuclei processed with a primary antibody targeting LMNB1 across a range of conditions, first with the pAG-EcoGII construct (FIG. 3). Total methylation was measured using a commercial colorimetric ELISA assay (MethylFlash from EpiGentek). Absorbance was measured on a microplate reader at 450 nm. This assay revealed that the DiMeLo-seq protocol was successfully methylating adenines, and the greatest level of methylation was achieved by increasing the primary antibody concentration dilution to 1:100, increasing the pAG-EcoGII to 7.3 μg, and performing pAG-EcoGII binding at room temperature.

Immunofluorescence imaging was also performed to qualitatively evaluate cell permeabilization, nuclear integrity, primary antibody on-target and background binding, and the effects of using a secondary antibody to recruit many methyltransferases to each primary antibody. For permeabilization in these experiments, alongside 0.02% digitonin, a different detergent, 0.5% NP-40, was tested which is frequently used in nuclear prep protocols. For detection of pAG-EcoGII binding, two different secondary antibodies were used: a goat anti-mouse IgG antibody that is not expected to bind to the rabbit primary or goat secondary antibodies but is expected to be bound by pAG, and a goat anti-V5 antibody expected to bind to the C-terminal V5 tag on pAG-EcoGII. These ensure that we are visualizing the pAG-EcoGII localization and not just the primary or secondary antibody localization.

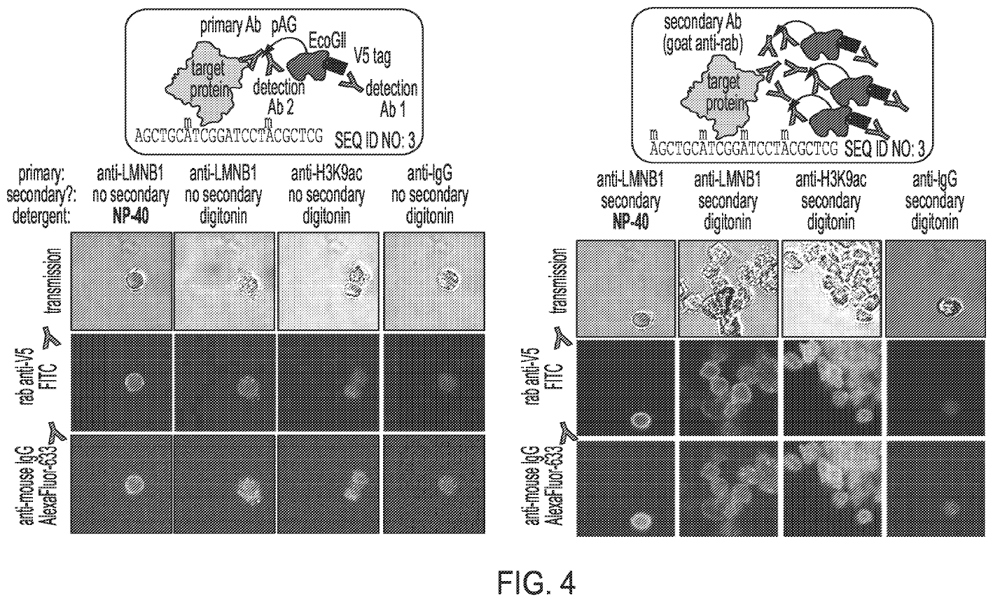

Confocal fluorescent images reveal that the pAG-EcoGII protein is able to diffuse into the permeabilized nucleus and localize correctly to the nuclear lamina (FIG. 4). The anti-LMNB1 samples show the expected ring patterns consistent with proper primary antibody binding to the nuclear lamina, which is not seen for an anti-H3K9ac antibody or an IgG isotype control. Both the transmission images and the anti-LMNB1 images would suggest NP-40 is better than digitonin at preserving nuclear integrity and removing cytoplasmic debris. Comparing fluorescence intensities confirms that there is an amplifying effect due to the use of secondary antibody. That is, it would appear that more molecules of pAG-EcoGII are recruited to the targeted regions when a secondary antibody is used, providing greater contrast between the nuclear lamina and the nuclear interior in the case of the anti-LMNB1 samples. The extent to which these imaging results are predictive of sequencing results was next investigated, as set out below.

### Sequencing Validation of DiMeLo-Seq and Comparison to Short-Read DamID-Seq

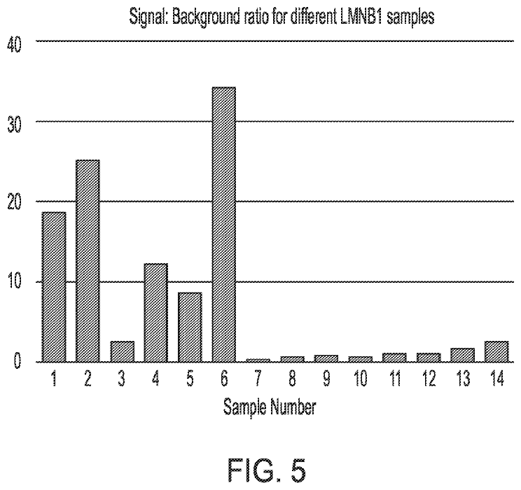

To validate that DiMeLo-seq was correctly targeting lamina-associated domains and producing sufficient signal by sequencing, DNA was sequenced from a range of different adenine-methylated samples using an Oxford Nanopore MinION sequencer with a v9.4 flowcell. High on-target and low off-target methylation was observed for all LMNB1-targeted samples and is not observed in controls (FIG. 5; samples 1-6 vs 7-14). For DNA methylated by Dam-LMNB1 or EcoGII-LMNB1 in vivo, long-read sequencing with direct methyladenine detection appeared to produce lower signal:background compared to short-read sequencing (samples 1-2 vs 3-4). In situ methylation with pAG-EcoGII yielded less signal than in vivo EcoGII-LMNB1 (sample 5 vs 4), but in situ methylation with pA-Hia5 exceeded the short-read, in vivo EcoGII-LMNB1 signal:background ratio significantly (sample 6 vs 2). This shows that DiMeLo-seq can provide robust signal in on-target regions while having low background.

| | | | | | | | short-read |
|---|---|---|---|---|---|---|---|
| | | | | | | | signal:back- |
| sample description | | | | | | long-read | ground |
| | sample | methyl- | methylation in | | | signal:background | coverage |
| type | number | transferase | situ or in vivo | target | readout | mA ratio | ratio |
| targeted | 1 | Dam | in vivo | LMNB1 | short-read DamID-seq | — | 18.639 |
| methylation | 2 | EcoGII | in vivo | LMNB1 | short-read DamID-seq | — | 25.327 |
| | 3 | Dam | in vivo | LMNB1 | long-read DiMeLo-seq | 2.6 | — |
| | 4 | EcoGII | in vivo | LMNB1 | long-read DiMeLo-seq | 12.4 | — |
| | 5 | pAG- | in situ | LMNB1 | long-read DiMeLo-seq | 8.7 | — |
| | | EcoGII | | | | | |
| | 6 | pA-Hia5 | in situ | LMNB1 | long-read DiMeLo-seq | 34.3 | — |
| control | 7 | Dam | in vivo | none | short-read DamID-seq | — | 0.325 |
| | 8 | EcoGII | in vivo | none | short-read DamID-seq | — | 0.726 |
| | 9 | EcoGII | in vivo | none | long-read DiMeLo-seq | 0.7 | — |
| | 10 | pAG- | in situ | none | long-read DiMeLo-seq | 0.6 | — |
| | | EcoGII | | | | | |
| | 11 | Hia5 | in situ | none | long-read DiMeLo-seq | 1.1 | — |
| | 12 | pAG- | in situ | IgG | long-read DiMeLo-seq | 1.0 | — |
| | | EcoGII | | | | | |
| | 13 | pA-Hia5 | in situ | IgG | long-read DiMeLo-seq | 1.6 | — |
| | 14 | pA-Hia5 | in situ | LMNB1 - | long-read DiMeLo-seq | 2.6 | — |
| | | | | no SAM | | | |

### Example 4

### Optimizing DiMeLo-Seq

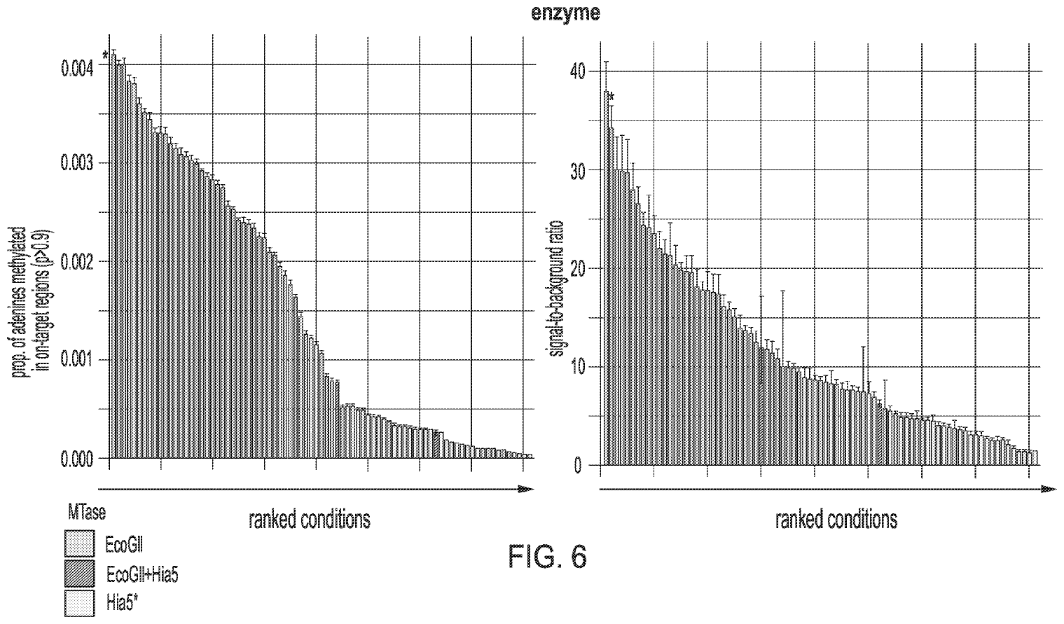

A rapid pipeline was next built to test over 80 different sequencing conditions for LMNB1-targeted DiMeLo-seq in HEK293T cells (FIG. 6). Herein and below, the combination of conditions that optimize specificity and sensitivity of DiMeLo-seq is provided. Conditions were selected that produced high rates of on-target methylation (in positive-control cLADs), with low rates of off-target methylation (in negative-control ciLADs). Over an order of magnitude improvement of performance was achieved compared to initial test conditions.

### Methyltransferase:

Hia5 in situ results in substantially more on-target methylation and higher signal:background compared to EcoGII, across a wide range of conditions (FIG. 6). This was surprising given their nearly identical performance in the in vitro assays.

### Detergent:

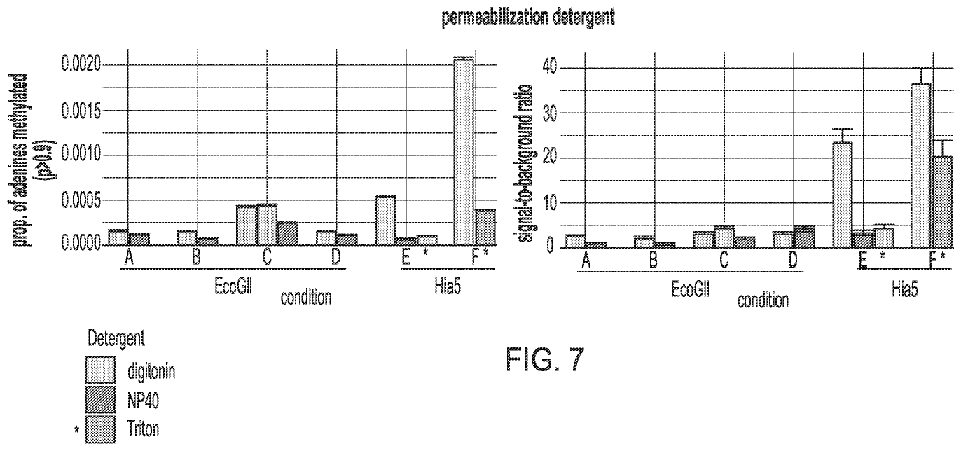

The permeabilization detergent has a critical impact on the performance of DiMeLo-seq. Although our immunofluorescence results suggested that NP-40 yielded better nuclear morphology and more recruitment of methyltransferase to the nuclear lamina (FIG. 4), the sequencing data showed the opposite: NP-40 resulted in inferior performance across multiple conditions, for both EcoGII and Hia5 (FIG. 7). This is especially surprising in light of the fact that the detergent is used only for 5 minutes at the start of the protocol, followed by hours of incubations and washes without it prior to enzyme activation—the detergent never touches the pA/G-MTase. The immunofluorescence data confirm that this is not due to a failure of permeabilization or a failure of antibody or pA/G binding (FIG. 4). This inhibitory effect of NP-40 was observed on both EcoGII and Hia5, but this effect has not been reported by others when NP-40 has been used for CUT&RUN (MNase enzyme) or CUT&Tag (Tn5 transposase). A similar effect was observed when 0.1% Triton X-100 was used (FIG. 7), but imaging was not used to formally rule out that this could be due to lower permeabilization efficiency rather than methylation inhibition (note: 0.1% Tween-20 is present in all of our wash buffers). NP-40 thus causes a change in the substrate chromatin that is not reversed by washing, and which specifically inhibits DNA methylation downstream in the protocol.

### Activation Buffer:

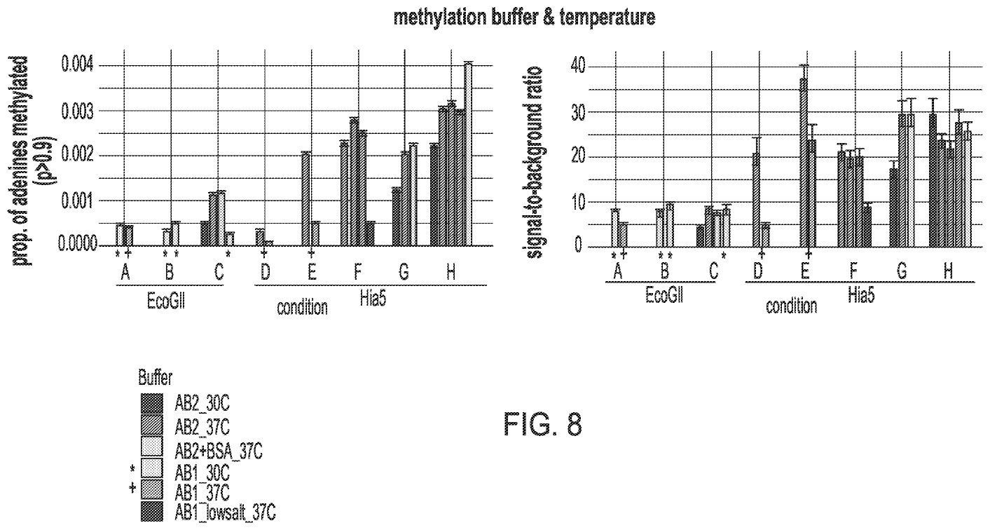

The choice of activation buffer and temperature in which the methylation step occurs has a critical impact on the performance of DiMeLo-seq (FIG. 8). Buffer AB1 performed worse than buffer AB2 (compositions described above) across all conditions. This was surprising given their similar performance in the in vitro methylation assay (FIG. 2). The inclusion of BSA in buffer AB2 improved its performance, consistent with the in vitro results, and it performed better in buffer AB2 at 37° C. than at 30° C.

### These Additional Factors Improved On-Target Methylation Rates and Signal-to-Background Ratios:

increased primary antibody concentration (1:50>1:100>1:500),
increased pA-Hia5 concentration (527 nM=200 nM>50 nM),
room temperature pA/G-MTase incubation (better than 4° C.),
increased SAM concentration during methylation (800 uM>500 uM).

### These Additional Factors Did not Improve Performance:

longer or shorter methylation incubation time (90, 60, 15 mins vs standard 30),
replenishment of SAM during methylation,
use of higher salt concentration (300 nM) in wash buffer (which has been reported to help in CUT&Tag protocols),
pre-treatment of DNA with RNAse to potentially increase DNA accessibility
use of a secondary antibody to recruit more pA-Hia5 molecules to the target region

### These Conditions Did not Appreciably Harm Performance:

using cells lightly fixed in 0.1% PFA for 2 mins,
using freshly thawed cells that were cryopreserved in DMSO-containing freezing medium and stored in liquid nitrogen (though anecdotally we observed shorter read lengths for these samples),
using concanavalin-A coated magnetic beads for cell processing (though this may limit capacity per tube and makes IF quality controls difficult),
starting with 5 million cells vs 1 million cells per tube (we observed some loss of performance with 10 million cells though).

### Example 5

### Mapping LMNB1-DNA Interactions in Human Centromeres

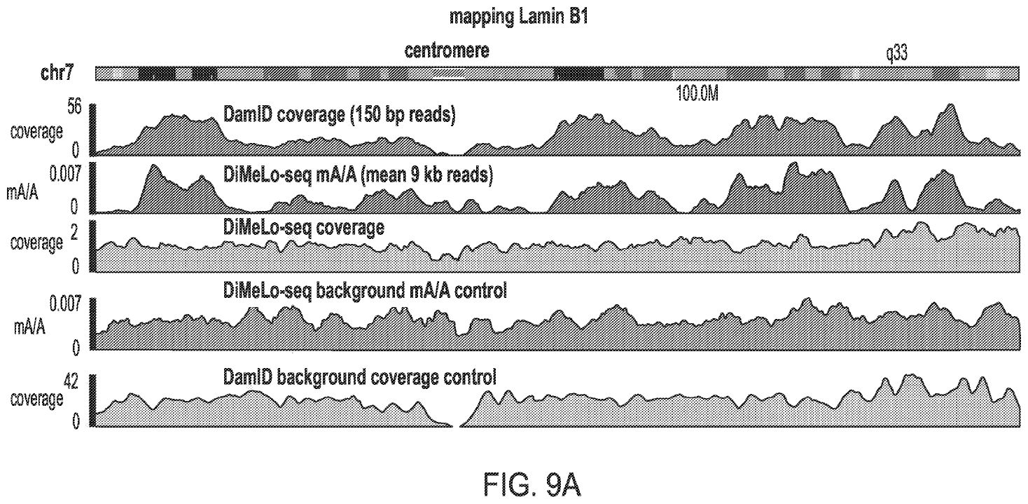

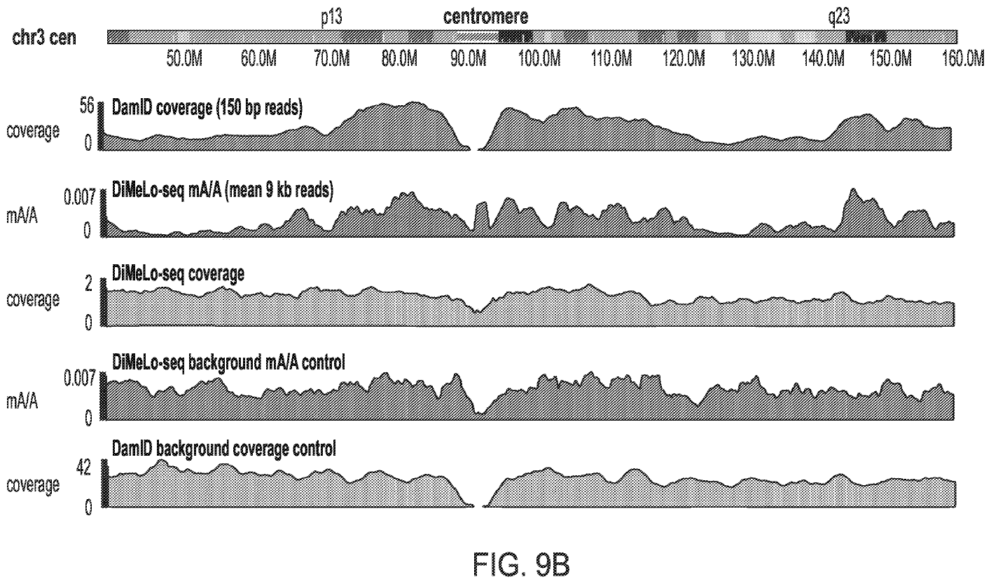

The combined results provided herein from the most optimal anti-LMNB1 conditions can be seen plotted along chr7 in FIG. 9A, and they correspond well with in vivo conventional DamID and in vivo DiMeLo-seq. Relatively uniform DiMeLo-seq coverage is also seen across the centromere given its long ONT reads, which is not the case for the short Illumina reads used in conventional DamID. The bottom two tracks show two background controls that indicate the relative accessibility, sequenceability, and mappability of each region of the genome. For DiMeLo-seq, this background control represents permeabilized nuclei that were treated with free-floating Hia5 (as in Fiber-seq, Stergachis, A. B., et al., (2020), Science, 368(6498), 1449-1454). For conventional DamID, this represents cells that expressed untethered Dam in vivo. In this chromosome's centromere, the advantage of long reads is clear, and it is also clear that the centromere is sufficiently accessible to be methylated by Hia5. The anti-LMNB1 methylation data suggest that this centromere is not strongly lamina associated, which aligns with broad observations that centromeres are often not preferentially associated with the nuclear lamina in mammalian cells, unlike certain other clades (reviewed by Hoskins, V. E., Smith, K. & Reddy, K. L., (2021), Curr Opin Genet Dev, 67, 163-173).

If the centromere of a different chromosome is examined, chromosome 3 (FIG. 9b), evidence of strong lamina association as well as a more pronounced dip in mappability and a dip in apparent accessibility at the centromere is seen. This underlines the need to produce longer reads at higher coverage in centromeric regions. However, this centromere does exhibit a robust signal of lamina association, apparent in the DiMeLo-seq data, which cannot be ascertained from the DamID data. This appears to be the strongest signal of lamina association at any centromere, and on close examination it appears that this may be related to the unusual nature of this centromere's organization. The alpha satellite of centromere 3 does not occur in one contiguous block but is divided into two pieces by a 2.5 Mb array of a different satellite DNA family, Human Satellite 1A (HSat1A), which is not known to be directly related to centromere function. Chromosome 4 has a similar centromeric organization, and it also appears to have a peak in lamina association in its own intervening HSat1A array, which diminishes inside the alpha satellite arrays.

### Example 6

Validating Modified Histone and CTCF Targeting with Nanopore Sequencing

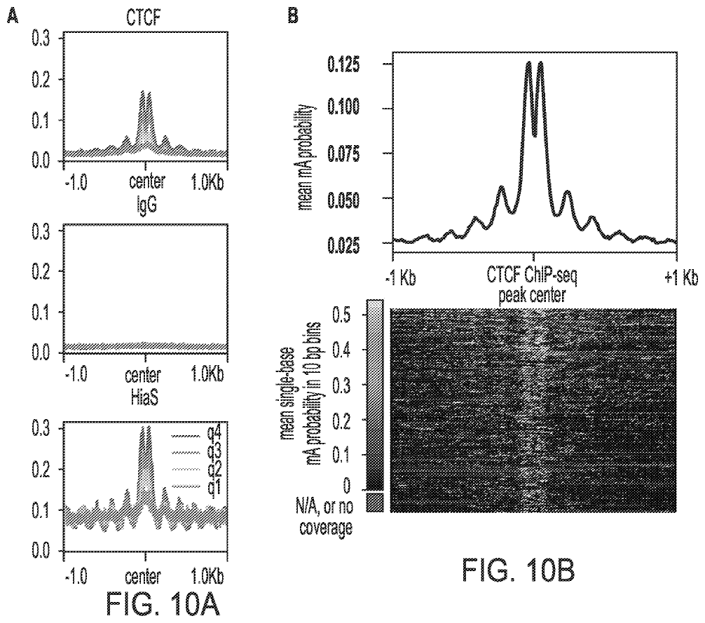

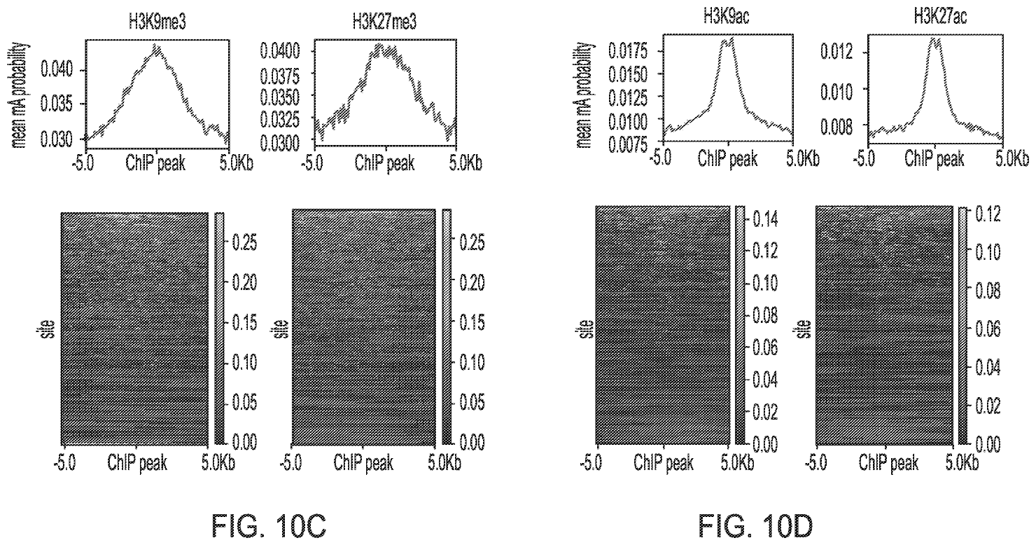

To further validate DiMeLo-seq, modified histones and CTCF in GM12878 cells were targeted and compared the results to published ChIP-seq data for the targets. The DiMeLo-seq experimental protocol was followed with 1:50 primary antibody dilution for each target. Targeted methylation is evident for each target. For CTCF, strong signal enrichment was seen at CTCF ChIP-seq peaks and observe nucleosome phasing (FIG. 10A). A similar signature is evident for the free-floating Hia5 condition, as both CTCF and free-floating Hia5 mark open chromatin. The IgG control does not show enrichment. To examine the spatial resolution achievable with DiMeLo-seq, methylation probability scores were plotted as a heat map, with rows corresponding to individual CTCF binding sites with the highest methylation probabilities in the surrounding 2 kb region (FIG. 10B). Because coverage is so low in this sample (1×), only 1 read is expected to overlap most of these peaks, providing a single-molecule view of methylation. It is clear that the reach of the methyltransferase decays completely to baseline around 500 bp from the peak center, but 60% of this decay happens within 100 bp. This sharp decay will help resolve the peak center much more finely as we begin to develop de novo peak calling algorithms for these data. These results show that DiMeLo-seq can provide single-molecule binding site resolution on the order of hundreds of base pairs, increasing the domain of proteins that can be usefully mapped with this method. DiMeLo-seq enrichment is also seen at respective ChIP-seq peaks for the histone marks H3K9me3, H3K9ac, H3K27me3, and H3K9ac (FIG. 10C).

### Example 7

### Enriching for Centromeric DNA

DiMeLo-seq does not involve any amplification steps, as it would not faithfully copy the DNA modifications from the native long DNA molecules. By default, DiMeLo-seq also contains no targeting or enrichment steps. If one is interested only in querying a particular subset of the genome, then sequencing the entire genome at high coverage can become costly and time consuming. DiMeLo-seq is particularly useful for examining repetitive regions of the genome like the alpha satellite repeat arrays that constitute functional centromeres. While there are several targeted nanopore sequencing methods that do not require amplification and thus are compatible with the DiMeLo-seq workflow, they are either not well suited to targeting large repetitive regions (Gilpatrick, T., et al., (2020), Nat Biotechnol, 38, 433-438), or they require advanced hardware to basecall and align reads in real time while rejecting off-target reads, which is likely to result in lower throughput (Kovaka S., et al., (2021), Nature Biotechnology, 39(4), 431-441).

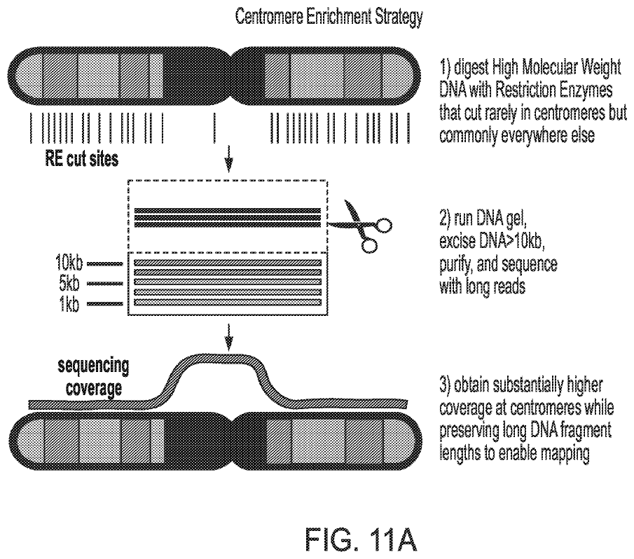

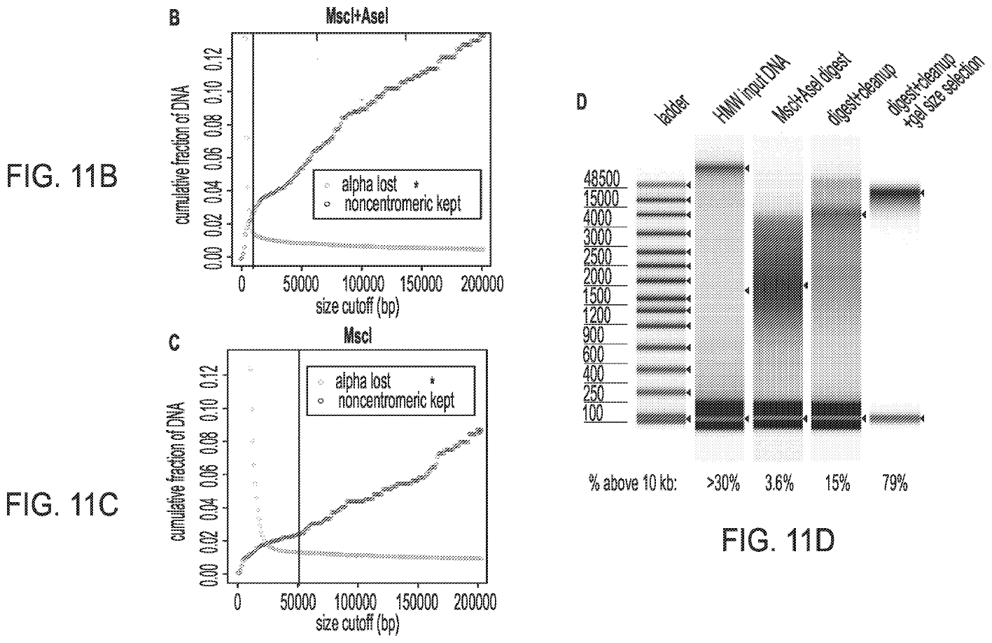

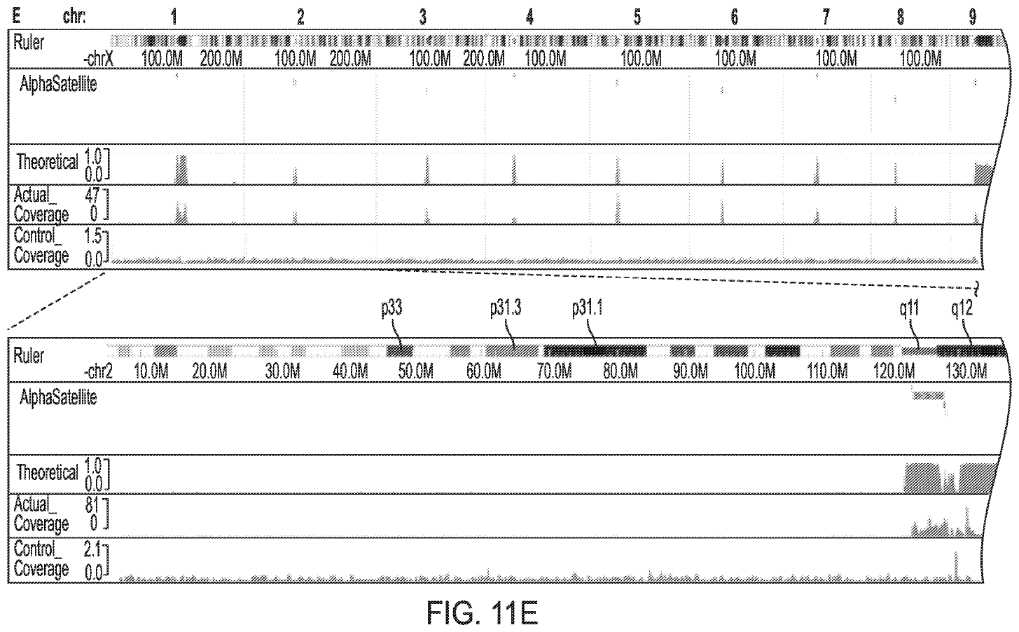

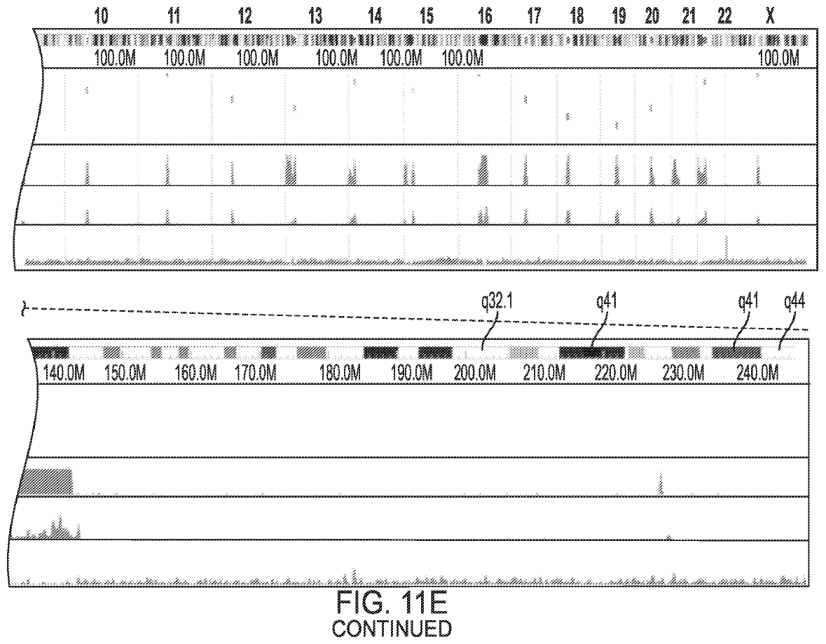

A method of enriching the input material itself for alpha satellite DNA was developed, along with a way to do it by leveraging the repetitive nature of satellites. Because satellite repeats are relatively short and homogeneous, short DNA k-mers (i.e., a DNA sequence's subsequence of length ‘k’) are not uniformly distributed throughout these regions. In fact, some k-mers are completely absent from some families of repeats; for example, GATC is missing from many large repetitive regions (Sobecki, M., et al., (2018), Cell Reports, 25, 2891-2903). Therefore one could digest the genome with a restriction enzyme that cuts motifs found commonly outside alpha satellite regions, but rarely inside them, in order to remove short digested DNA fragments by size selection, leaving mostly long, undigested alpha satellite DNA (FIG. 11A).

To validate the feasibility of this approach, digestion of the T2T chm13 reference sequence was simulated with a set of all restriction enzymes available from New England Biolabs that had 4-6 bp cut sites and that were annotated as being insensitive to CpG or Dam methylation. Of those, 28 enzymes were selected for which fewer than 5% of fragments mapped to alpha satellite, and for which the genome was digested into at least 200,000 total fragments. All simulated fragments under 10 kb were then removed, to simulate a size selection process, and the fraction of remaining fragments mapping to centromeres were determined. This allowed an estimate of the theoretical enrichment of centromeric sequences as well as the systematic loss of centromeric sequences predicted to be digested into fragments under the size cutoff. Double and triple digest combinations of the top 3 enzymes were tested with >4-fold alpha satellite enrichment and <3% alpha satellite loss (MscI, AscI, PvuII), and the best possible overall enrichment regime was found to be a double digest with MscI and AseI, predicted to yield 18-fold enrichment of alpha satellite with only 0.8% systematic loss of alpha satellite (FIG. 11B).

To test this approach, ˜100 μg high-molecular-weight (HMW) DNA isolated from ˜25M HEK293T cells was digested overnight with MscI and AseI, then cleaned up the digest with a column that depletes fragments under 3 kb (Zymo gDNA Clean & Concentrator Kit), yielding 15 μg. Early attempts to perform size selection with a Circulomics Short Read Eliminator XS kit resulted in extremely low yields. Instead, a 0.3% TAE+agarose gel (using SeaKem Gold agarose, which is specialized for large fragment separations) was loaded and run at low voltage (2 V/cm) until fragments under 10 kb were visibly separated from a visible HMW band (˜1 h). Everything above 10 kb was cut out, including the loading well, and purified the DNA using a Zymo Large DNA Fragment Recovery kit. This yielded ˜1.8 μg of DNA, which was library prepped and sequenced on a MinION device. By mapping reads back to the reference sequence, ˜20-fold enrichment of alpha satellite sequences was observed (FIG. 11E). Specifically, while alpha satellite higher order repeats constitute only 2.3% of the genome, reads overlapping these regions represented 46.2% of bases on all mapped reads. This means a single 72 hour, <$1500, 20 Gb run on a MinION flowcell could yield ˜130× coverage of alpha satellite regions, which is enough to split over many DiMeLo-seq samples. Without enrichment, obtaining this same coverage on a single MinION would require 2 months and $30 k. Preserving longer centromeric DNA fragments is likely possible by using a single restriction enzyme digestion followed by electroclution of large DNA fragments from the gel slice.

### Example 8

### Measuring Haplotype-Specific Protein-DNA Interactions and CpG Methylation

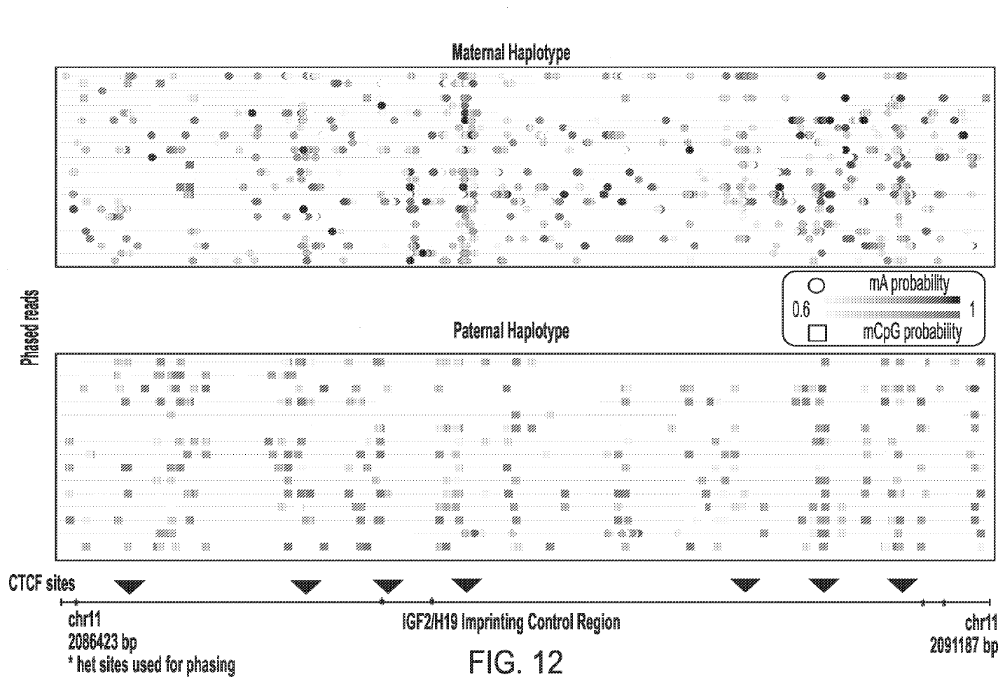

DiMeLo-seq can measure the effect of haplotype-specific genetic or epigenetic variation on protein binding. The ability to map haplotype-specific interactions is useful in studying imprinted genomic regions such as the IGF2/H19 Imprinting Control Region, where CpG methylation on the paternal allele prevents CTCF binding, while on the maternal allele, CTCF is able to bind (FIG. 12). FIG. 12 also demonstrates the ability to capture joint information about endogenous CpG methylation and protein-DNA interactions on the same long single molecules. Multiple binding sites are spanned by single molecules, highlighting the ability to detect joint long-range binding information from the same chromatin fibers as well.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of determining the genomic location of at least one biomolecule-genomic DNA interaction, said method comprising the steps of:
(a) incubating a biomolecule of interest under conditions that allow the biomolecule of interest to contact a genomic DNA sequence;
(b) isolating and permeabilizing nuclei from the cells in (a) under conditions that allow isolation of genomic DNA bound by the biomolecule of interest;
(c) contacting the biomolecule bound to genomic DNA with a first binding moiety capable of specifically binding to the biomolecule of interest;
(d) contacting the first binding moiety with a second binding moiety capable of specifically binding to the first binding moiety, wherein said second binding moiety is conjugated to an enzyme capable of modifying genomic DNA;
(e) incubating the first binding moiety and second binding moiety of (d) under conditions that allow modification of genomic DNA;
(f) isolating and preparing the genomic DNA for sequencing, wherein said preparing does not require amplification of the DNA; and
(g) sequencing the genomic DNA under conditions that allow determining the location of the biomolecule-DNA interaction.

2. The method of claim 1, wherein the enzyme capable of modifying genomic DNA is a DNA methyltransferase.

3. The method of claim 2, wherein the DNA methyltransferase is selected from the group consisting of DNA adenine methyltransferase (Dam) or a biologically active fragment thereof, (ii) EcoGII methyltransferase or a biologically active fragment thereof, Hia5 or a biologically active fragment thereof, M.CviPI or a biologically active fragment thereof, and M.SssI or a biologically active fragment thereof.

4. The method of claim 3 wherein the DNA methyltransferase is Hia5 or a biologically active fragment thereof.

5. The method of any one of claims 1-4, wherein the sequencing conditions of step (g) allow sequencing of more than approximately 1,000 base pairs (bp) in a single sequencing read.

6. The method of any one of claim 1-5, wherein the interaction is in a cell and the incubating of step (a) comprises incubating a collection of cells.

7. The method of any one of claims 1-6, wherein the first binding moiety is an antibody.

8. The method of any one of claims 1-7, wherein the second binding moiety is an antibody, nanobody, single-chain variable fragment (scFv) protein-A, protein-G, or protein-A/G.

9. The method of claim 6, wherein the cell is selected from the group consisting of a bacterial cell, a eukaryotic cell, prokaryotic cell, an archaeal cell and a virus.

10. The method of claim 9, wherein the cell is a mammalian cell.

11. The method of claim 10, wherein the cell is a human cell.

12. The method of any one of claims 1-11, wherein the biomolecule of interest is selected from the group consisting of a protein, a RNA, and a RNA-DNA hybrid.

13. The method of claim 12, wherein the biomolecule is a RNA selected from the group consisting of ncRNA, tRNA, rRNA, snRNA, snoRNA, miRNA, mRNA, and TERC.

14. The method of claim 12, wherein the biomolecule is a protein selected from the group consisting of a nuclear lamina protein, a nucleolar protein, a transcription factor, a histone or histone variant, centromere protein A, an intracellular scFV, a chromatin-modifying enzyme, an RNA polymerase, a DNA polymerase, a DNA helicase, a DNA repair protein, a Cas9 protein, a dCas9 protein, a zinc finger protein, a TALE protein, a CTCF protein, a cohesion protein, a synaptonemal complex protein, a telomere-binding protein, a centromere-binding protein, an outer kinetochore protein, a splicing protein and a chromatin remodeling protein.

15. The method of claim 6, wherein the collection of cells are induced to express the protein of interest.

16. The method of claim 15, wherein the protein of interest is a recombinant protein and is expressed from an expression vector.

17. The method of any one of claims 1-16, wherein 2, 3, 4, 5, 6, 7, 8, 9 or 10 or more biomolecule-genomic DNA interactions are determined.

18. The method of any one of claims 1-17, wherein the modifying genomic DNA of step (e) comprises modifying one or more nucleotides at one or more locations selected from the group consisting of (a) within 1-50 nucleotides of the genomic DNA binding site of the biomolecule, (b) topologically near the genomic DNA binding site of the biomolecule, and (c) both (a) and (b).

19. The method of any one of claims 1-18, wherein the isolating and permeabilizing nuclei of step (b) comprises contacting nuclei with digitonin.

20. The method of any one of claims 1-7 and 9-19, wherein the contacting the second binding moiety of step (d) is protein-A, protein-G, or protein-A/G.

21. The method of any one of claims 1-20, wherein the incubating of step (e) comprises incubating in the presence of bovine serum albumin (BSA) and low salt conditions.

22. The method of any one of claims 1-21, wherein the isolating and preparing the genomic DNA for sequencing of step (f) comprises high molecular weight DNA extraction.

23. The method of any one of claims 1-12, wherein the sequencing of step (g) comprises long read sequencing.
